(19) United States
(12) Patent Application Publication
(10) Pub. No.: US 2025/0111927 A1
(43) Pub. Date: Apr. 3, 2025

(54) INFORMATION PROCESSING SYSTEM, INFORMATION PROCESSING METHOD, AND NON-TRANSITORY COMPUTER-READABLE MEDIUM STORING PROGRAM

(71) Applicant: CureApp, Inc., Tokyo (JP)

(72) Inventor: Hikaru TAKEMURA, Sunnyvale, CA (US)

(73) Assignee: CureApp, Inc., Tokyo (JP)

(21) Appl. No.: 18/817,860

(22) Filed: Aug. 28, 2024

Related U.S. Application Data
(60) Provisional application No. 63/541,118, filed on Sep. 28, 2023.

(51) Int. Cl. G16H 20/60; G16H 10/60

(52) U.S. Cl. CPC G16H 20/60; G16H 10/60

(57) ABSTRACT

A technology to support effective use of information collected by different lifestyle improvement support application is provided. An information processing system includes at least one processor, wherein the at least one processor acquires first information related to lifestyle collected through a first lifestyle improvement support application, and transfers the first information to second information, based on corresponding relationship between the acquired first information and the second information used in a second lifestyle improvement support application.

### CROSS REFERENCE TO RELATED APPLICATIONS

This application is based on and claims priority under 35 USC § 119 to U.S. provisional patent application Ser. No. 63/541,118, filed Sep. 28, 2023, the disclosure of which is incorporated herein by reference in its entirety.

### BACKGROUND

### Technical Field

The present disclosure relates to an information processing system, an information processing method, and a non-transitory computer-readable medium storing a program.

### Related Art

Currently, companies in the United States are increasingly interested in Digital Therapeutics (DTx), which uses digital technologies to support prevention, diagnosis, and therapy of disease, and efforts are being made to reduce the prevalence of lifestyle-related diseases.

On the other hand, the needs of companies for DTx are not always the same. Therefore, various types of DTx applications (hereinafter referred to as “lifestyle improvement support app”) can be developed remaining in a state that handled data is not standardized. However, in a method how to collect information related to an employees' lifestyle from scratch at a job change destination company, there is a time lag in supporting improvement of the employee's lifestyle.

Aspects of the present disclosure provide a technology to support effective use of information collected by a different lifestyle improvement support app.

### SUMMARY

An aspect of the present disclosure provides an information processing system including at least one processor, wherein the at least one processor acquires first information related to lifestyle collected through a first lifestyle improvement support application, and transfers the first information to second information, based on corresponding relationship between the acquired first information and the second information used in a second lifestyle improvement support application.

Another aspect of the present disclosure provides an information processing method including: acquiring first information related to lifestyle collected through a first lifestyle improvement support application, and transferring the first information to second information, based on corresponding relationship between the acquired first information and the second information used in a second lifestyle improvement support application.

Still another aspect of the present disclosure provides a non-transitory computer readable medium storing a program that causes a computer to execute an information processing method including acquiring first information related to lifestyle collected through a first lifestyle improvement support application, and transferring the first information to second information, based on corresponding relationship between the acquired first information and the second information used in second lifestyle improvement support application.

According to the aspects of the present disclosure, it is possible to support effective use of information collected by a different lifestyle improvement support app.

### BRIEF DESCRIPTION OF DRAWINGS

FIG. 1 illustrates a configuration example of an information processing system assumed in Exemplary embodiment 1;

FIG. 2 illustrates a hardware configuration of a support system;

FIG. 3 illustrates a cause of transferring user data assumed in Exemplary embodiment 1;

FIG. 4 is a processing sequence diagram illustrating a process of transferring user data in an information processing system;

FIG. 5 shows an example of a new registration screen shown on a display of user's information terminal;

FIG. 6 is a sequence diagram illustrating an overview of a process of registration/a process of support;

FIG. 7 shows an example of a conversion of expression executed in the process of transferring;

FIG. 8 illustrates a cause of transferring user data assumed in Exemplary embodiment 2;

FIG. 9 illustrates a cause of transferring user data assumed in Exemplary embodiment 3;

FIG. 10 is a flowchart illustrating a process of transferring user data, which is in holding state.

### DETAILED DESCRIPTION

Hereinafter, exemplary embodiments of the present disclosure will be described with reference to the drawings.

### Exemplary Embodiment 1

### <System Configuration>

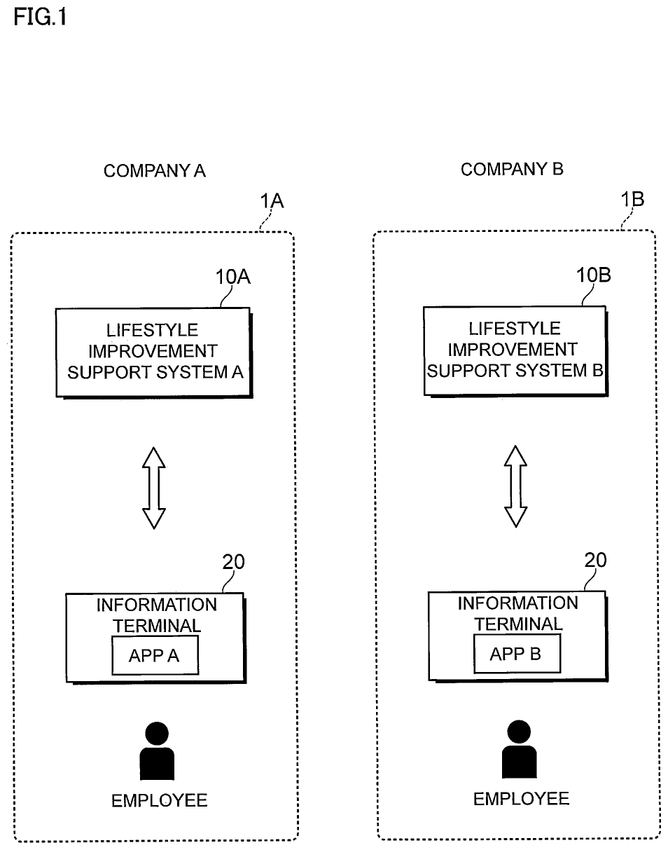

FIG. 1 illustrates a configuration example of an information processing system 1A,1B assumed in Exemplary embodiment 1.

The information processing system 1A is a system that is operated by a company A or an enterprise entrusted by the company A (hereinafter referred to as “company A”), and the information processing system 1B is a system that is operated by a company B or an enterprise entrusted by the company B (hereinafter referred to as “company B”).

In the case of the exemplary embodiment, the company A and company B are independent of each other both in terms of personnel and capital. Therefore, the information processing system 1A and the information processing system 1B are operated independently each other.

The information processing system 1A is configured with a lifestyle improvement support system 10A (hereinafter referred to as “support system 10A”) which provides support service A (hereinafter referred to as “support service A”), an information terminal 20 operated by an employee of the company A, and a not-shown network N that connects above these.

On the other hand, the information processing system 1B is configured with a lifestyle improvement support system 10B (hereinafter referred to as “support system 10B”) which provides support service B (hereinafter referred to as “support service B”), an information terminal 20 operated by an employee of the company B, and a not-shown network N that connects above these.

In the case of the exemplary embodiment, between the support service A and the support service B, at least one of substance of service or handled data is different.

The support system 10A distributes an app A that supports improvement of lifestyle (hereinafter referred to as “support app A”), stores user data entered through the support app, provides information to support lifestyle improvement.

On the other hand, the support system 10B distributes an app B that supports improvement of lifestyle (hereinafter referred to as “support app B”), stores user data entered through the support app, provides information to support lifestyle improvement.

The support app A is installed in the information terminal 20 operated by an employee of the company A, and used for a record of daily life of the employee.

The support app B is installed in the information terminal 20 operated by an employee of the company B, and used for a record of daily life of the employee.

A record of daily life includes, for example, blood pressure values, weight, pulse, history of activity, and a record of feelings and moods.

Hereinafter, in case that the support service A and the support service B are not distinguished, it is simply expressed as the support service.

Similarly, in case that the support app A and the support app B are not distinguished, it is expressed as the support app.

Similarly, in case that the support system 10A and the support system 10B are not distinguished, it is expressed as the support system 10.

The information terminal 20 is a so-called computer terminal. The information terminal 20 in the exemplary embodiment is, for example, a smart phone, a tablet-type computer, a laptop-type computer, and a desktop-type computer.

The information terminal 20 can communicate with the support system 10 operated by the company where the user works through the network N which is not shown.

The information terminal 20 uploads the user data entered through the support app to the corresponding support system 10 respectively, and receives the support services from the support system 10.

The network N is, for example, a local area network (LAN), the Internet, or a mobile communication system (4G, 5G).

### <Hardware Configuration of System>

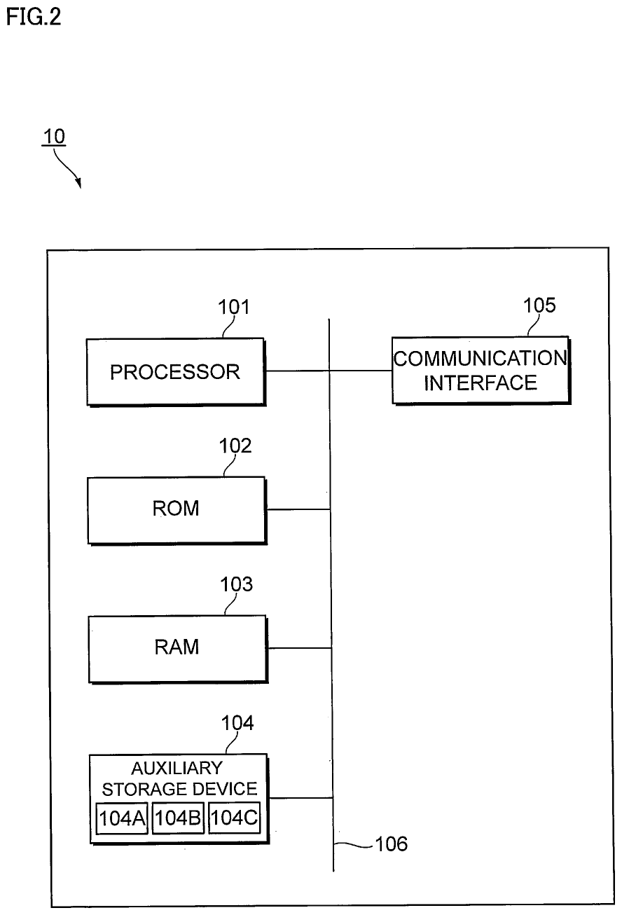

FIG. 2 illustrates a hardware configuration of the support system 10. The support system 10 is configured with, for example, one or more servers.

The support system 10 includes a processor 101, a Read Only Memory (ROM) 102 storing a Basic Input Output System (BIOS), etc., a Random Access Memory (RAM) 103 used as a work area of the processor 101, an auxiliary storage device 104, and a communication interface 105. Each device is connected via a bus and other signal wires 106.

The processor 101 is a device that executes various kinds of functions through the execution of programs. In the exemplary embodiment, the lifestyle improvement support app and Operating System (OS) are collectively called programs.

The processor 101, ROM 102, and RAM 103 function as a computer.

The auxiliary storage device 104 is configured with, for example, a hard disk device or a semiconductor storage.

The communication interface 105 is an interface for communicating with other servers or user terminals 20 (refer to FIG. 1) via the network N. The communication interface 105 complies with Ethernet (registered trademark), Wi-Fi (registered trademark), mobile communication systems and other communication standards.

### <Data Stored in Auxiliary Storage Device>

The auxiliary storage device 104 stores a support app 104A, a user data 104B collected through the support app 104A and a table 104C used to transfer the user data (hereinafter referred to as “user data transferring table”).

### <Support App>

The support app 104A is an app that corresponds to the support service which each company adopts.

The support app 104A includes a server app that is executed on the support system 10 and a client application that is executed on the information terminal 20 operated by the employee.

### <User Data>

The user data 104B includes, for example, user attributes (gender, date of birth, age, height, weight), blood pressure values, activity records, mood records, physical condition records, medical examination history, operation history of the lifestyle improvement support app, biological characteristics, psychological characteristics, social characteristics, habits, goal achievement status, featured categories presented by the lifestyle improvement support services, recommended actions for featured categories, a content that encourages the practice of the actions (including information that identifies the content), information that motivates the continuous practice of the action, record of proficiency with the content, etc. However, the user data does not need to be all of such information, but may be part thereof.

The blood pressure values are, for example, systolic blood pressure (i.e., the highest blood pressure value) and diastolic blood pressure (i.e., the lowest blood pressure value).

The activity records include, for example, usage history of the support app, medications records, dietary records, smoking records, drinking records, and exercise records. The activity records are an example of information about activities.

The medications records include, for example, the substance and number of medications taken, and the time at which the medications are taken. The medications records are an example of information about medications.

The dietary records include, for example, the substance of the meals consumed, and the amount of salt taken. The dietary records are an example of information about diet.

The smoking records include, for example, the presence or absence of smoking, the date and time of smoking, and the number of cigarettes smoked. The smoking records are an example of information about smoking.

The drinking records include, for example, the date and time of drinking, the amount of alcohol intake, and the type of alcohol. The drinking records are an example of information about drinking.

The exercise records include, for example, records related to sports as well as records of activities in daily life such as walking, shopping, and cleaning. The exercise records are an example of information about exercise.

The mood records include, for example, a perceived mood. The mood records are an example of information about mood.

The physical condition records include, for example, a perceived physical condition or symptoms. The physical condition records are an example of information about physical condition.

The medical examination history includes, for example, the start date of the therapy, the date of the consultation, the details of the therapy, and advice from the doctor or other healthcare professionals. The medical examination history is an example of information about medical examination.

The operation history of the support app includes, for example, the operation history related to the activation operation and the entry operation of blood pressure values and reflection.

The biological characteristics include, for example, the presence or absence of other diseases, the presence or absence of injuries during therapy, the presence or absence of knee or foot pain, the presence or absence of experience in hypertensive therapy, the number of years since the high blood pressure was pointed out, the intensity of seasoning at home, and the amount of intake of food.

The psychological characteristics include, for example, expectations for the support app, willingness to acquire knowledge and information that can contribute to improve lifestyle, feeling that salt reduction is challenging, feeling inability to alter the sense of taste, and a psychological resistance to leaving meals.

The social characteristics include, for example, wake time, bedtime, work pattern (such as shifts, day duty, and night duty), days of the week to work, the start time of the work, the time to return home, regular days off, and the presence or absence of a heater in a locker room.

The habits include, for example, an exercise habit, a weight measurement habit, a habit of calorie checking on food labels, a habit of low-fat food choices, a habit of not taking caffeine after 4 p.m., a habit of eating and drinking after 10 p.m., a habit of skipping breakfast, a habit of eating between meals, a habit of bathing one hour before bedtime, a habit of stretching and massaging before bedtime, and a habit of sleeping for more than six hours.

The goal achievement status includes, for example, the relationship between the user's status and the goal set by the doctor, etc. for each user.

Note that the above-described information can be classified into subjective information and objective information.

The featured categories presented by the support service are, for example, categories from among diet, exercise, weight loss, sleep, stress, and alcohol that have a high possibility of contributing to the improvement of the target user's lifestyle (, for example, 3 categories). Note that the featured categories are determined based on, for example, answers to questions to the target user regarding his/her lifestyle habits and preferences, and notified to the target user by the support service. Review and update of the featured categories are carried out periodically (for example, every month) in response to the status of lifestyle improvements.

The recommended actions for featured categories (hereinafter also referred to as “recommended actions”) are actions selected from a group of candidates for actions related to a featured category. For example, in the case where the featured category is “exercise,” the candidates for the actions include walking (total distance, total length of time, etc.), jogging (distance, total length of time, etc.), housework (substance, length of time by substance, total length of time, etc.), cycling (total distance, total length of time, etc.), swimming (total distance, total length of time, etc.), ball games (type, total length of time, etc.), and dance (total length of time).

The recommended actions are determined based on a history of practice by the target user and a record of improvement effects after practice, etc. and are notified from the support app to the target user. Note that the recommended actions may be selected by the target user. The review and update of the recommended actions are carried out in a shorter cycle (for example, every week) than the review and update of the featured categories.

The content that encourages the practice of the actions (including information that identifies the content) refer to how to practice recommended actions and other learning content.

The information that motivates the continuous practice of the action is, for example, points or other rewards provided for the continued practice of the target user. This type of information includes, for example, accumulated point increases, object advancement, growth, and evolution, etc.

The proficiency with the content is the target user's level of understanding as evaluated through the target user's responses and actual performance. The proficiency is evaluated in the lifestyle improvement support service and notified to the target user.

The user data 104B in the exemplary embodiment includes user data collected from employee, in relation to other support service, in addition to the user data collected from employee, in relation to support service currently in operation.

Note that, the user data collected from employee in relation to other support service is used after being adjusted to (being transferred and processed) data format, expression, etc. of the support service currently in operation.

### <User Data Transferring Table>

The user data transferring table 104C is a table that is used for a case when user data that an employee is stored by using other support app is transferred to service which his/her company operates.

The user data transferring table 104C stores the corresponding relationship between the attributes of the transferring source and those of the transferring destination. In the case of the exemplary embodiment, the user data transfer table 104C stores not only corresponding relationship of the attribute names, but also the processing rules at the time of transferring and the processing rules after transferring.

Corresponding relationship of attribute name includes, for example, the relationship associating between “weight” and “body weight”, and the relationship associating between “weight” and “body weight”.

The processing rules at the time of transferring include, for example, conversion rules for display format, units, etc.

The processing rules after transferring include, for example, processing rules which are necessary for the continuous provision of services using the transferred user data.

Note that, the user data transferring table 104C is prepared according to the other support apps which are the transferring sources.

### <Process of Transferring>

### <Timing of Execution>

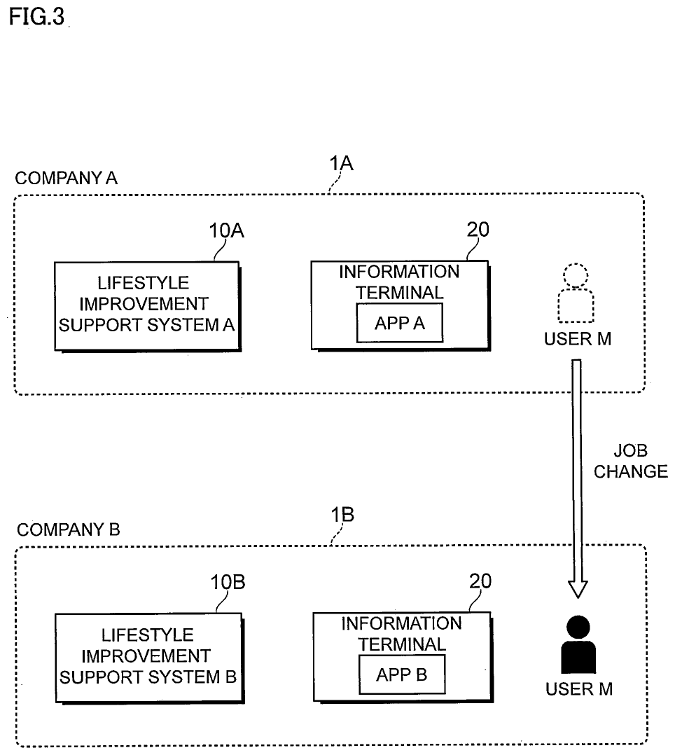

FIG. 3 illustrates a cause of transferring user data assumed in Exemplary embodiment 1. In FIG. 3, components corresponding to those of FIG. 1 are provided with same reference signs.

The example shown in FIG. 3 represents a scene where a user M changes job from company A to company B. Both company A and company B adopt the lifestyle improvement support service.

Therefore, the user M has been received provision of the service of a support service A by using an app A, while the user M has been the employee of the company A.

However, the company B that is the user M's job change destination, a support service B is adopted.

Therefore, the user M after changing his/her job installs the support app B provided by a support system 10B of the company B in an information terminal 20, and prepares an environment to receive provision of the support service B from the support system 10B.

When the environment is prepared, the support service B starts collecting the user M's user data through the support app B.

In the exemplary embodiment, when the new support app B is installed, a process of transferring of user data related to the support app A.

In the process of transferring, for example, the user data of the support app A is uploaded from the information terminal 20 to the support system 10B. Note that, with the user M's consent, the support system 10B may be possible to acquire directly the user M's user data stored in the support system A.

### <Specific Example of a Process of Transferring>

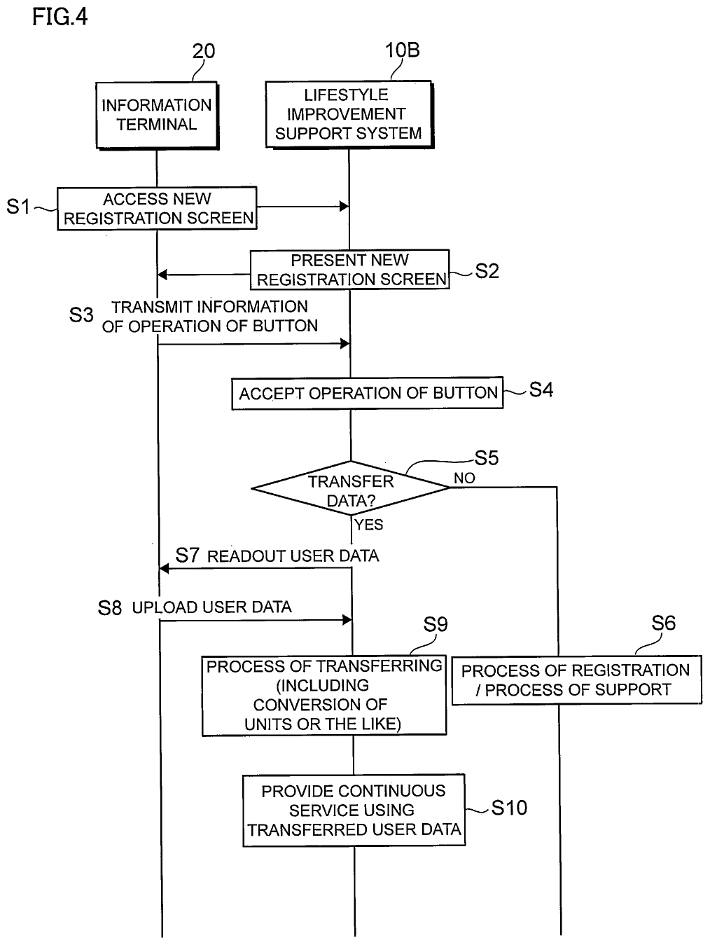

FIG. 4 is a processing sequence diagram illustrating a process of transferring user data in the support system 10B.

The processing sequence shown in FIG. 4 is executed through the execution of program by the processor 101 (refer to FIG. 2) which configures the support system 10B that is user M's (refer to FIG. 3) job change destination. Note that, the reference sign S shown in the figure represents a step.

### [Step 1]

The process of transferring shown in FIG. 4 is started through the user M's operation. Firstly, the user M accesses a new registration screen for an employee, which the support system 10B discloses, through the operation of the information terminal 20. Note that, necessary information for access is provided from the job change destination to the user M in advance.

### [Step 2]

When the support system 10B detects access to the new registration screen of the support service B, the support system 10B presents the new registration screen on the user M's information terminal 20, which is a source of access. Note that, the information terminal 20 has a configuration in which a touch panel (display and electrostatic touch sensor) and a GPS receiving section that receives global positioning system (GPS) radio waves are added to the hardware shown in FIG. 2.

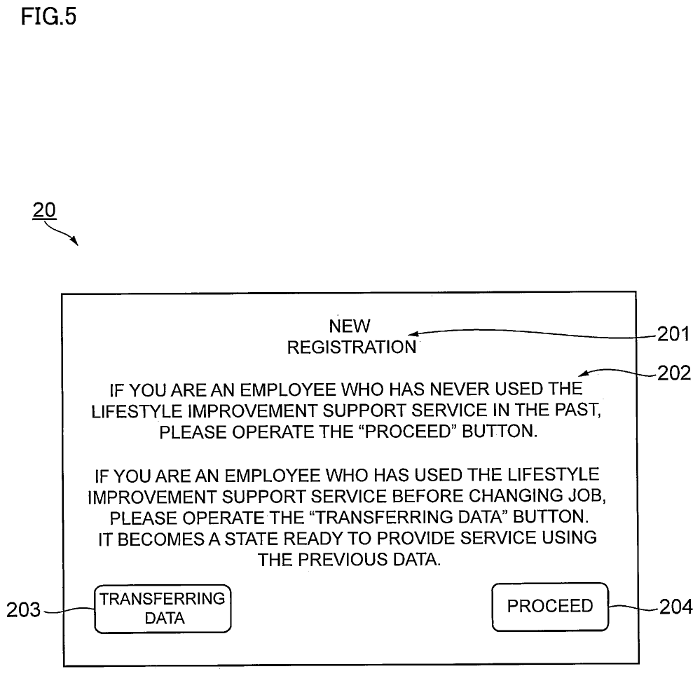

FIG. 5 shows an example of a new registration screen shown on the display of user M's information terminal 20.

The new registration screen shown in FIG. 5 includes a “New registration” title 201, an operation instruction 202, a “Transferring data” button 203, and a “Proceed” button 204.

In case of FIG. 5, in the operation instruction 202, there are descriptions which say “If you are an employee who has never used the lifestyle improvement support service in the past, please operate the “Proceed” button.”, “If you are an employee who has used the lifestyle improvement support service before changing job, please operate the “Transferring data” button.” and “It becomes to be possible to provide service using the previous data.”

The first sentence is a description sentence for an inexperienced person of the support service, and the second and third sentences are description sentences for an experienced person of the support service.

Note that, “Transferring data” button 203, and a “Proceed” button 204 are so-called icon buttons. When the user M operates each button, the procession of label that is attached to the operated button is started.

### [Step 3]

Return to the description of FIG. 4.

The information terminal 20 transmits the information of accepted operation of the button to the support system 10B.

### [Step 4]

The support system 10B accepts the operation of the button.

### [Step 5]

The support system 10B determines whether the operation of the button is the transferring data.

### [Step 6]

When the determination in step 5 is a negative result (i.e., in case of the operation of the “Proceed” button 204 (refer to FIG. 5)), the support system 10B performs a process of registration/a process of support.

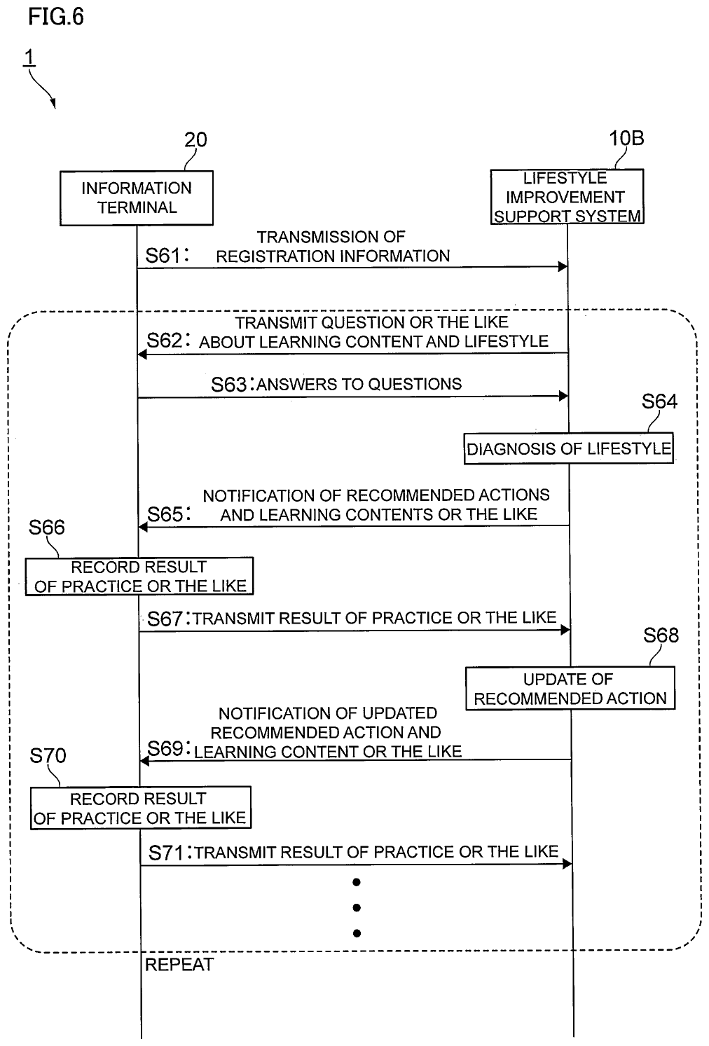

FIG. 6 is a sequence diagram illustrating an overview of a process of registration/a process of support.

### [Step 61]

In FIG. 6, the registration information of the user M is transmitted from the information terminal 20 to the support system 10B. For example, measurement values of blood pressure and pulse, information of taken medicine or the like, are transmitted as the registration information. In the exemplary embodiment, the input and transmission of the registration information is executed through the support app.

### [Step 62]

The support system 10B transmits questions or the like about learning content and lifestyle to the information terminal 20.

The learning content includes knowledge and information that contribute to the improvement of lifestyle. The learning content is prepared for each disease, for example. In the case of the exemplary embodiment, high blood pressure is assumed as a disease. Note that the learning content may be text-based, static-image-based, moving-image-based, or speech-based.

The questions related to the lifestyle are questions about the habits and preferences in daily life of each user. The questions in the exemplary embodiment are prepared to include, for example, six categories of diet, exercise, weight loss, sleep, stress, and alcohol.

### [Step 63]

The information terminal 20 transmits an answer to the question from user M to the support system 10B. Note that, for example, the answer to the question is entered through the selection of options by the user.

### [Step 64]

The support system 10B diagnoses the lifestyle of the target user based on the received answers. In the case of the exemplary embodiment, for example, scores of the target user is calculated for each of the above-described six categories. In the case of the exemplary embodiment, the three categories of the low score are extracted as the featured categories which are likely to contribute to improve lifestyle. Note that, the extracted featured categories are notified as a diagnoses result to the user.

### [Step 65]

The support system 10B notifies the information terminal 20 of the recommended actions and the learning content or the like related to the featured categories. The recommended action here is an action that contribute to improve the featured categories. However, it is not necessary to notify one recommended action per one featured category, and more than two recommended actions per one featured category may be notified.

In the case of the exemplary embodiment, the support system 10B determines the recommended action which is notified to the user M, however, the mechanism that the user chooses the action to be practiced by his/herself may be adopted.

### [Step 66]

The information terminal 20 records such as the result of practice for the recommended action and the measurement result of blood pressure value or the like in the auxiliary storage device 104 (see FIG. 2). In the case of the exemplary embodiment, the result of practice for the recommended action and the measurement value are input through the support app B. Note that, the result of practice and the measurement result or the like are recorded as user data in the auxiliary storage device 104.

### [Step 67]

The information terminal 20 transmits the recorded result of practice or the like to the support system 10B. The transmission of the result of practice or the like may be performed on each input of the user data, and also may be performed at the timing of access to the support system 10B by the user.

### [Step 68]

The support system 10B updates the recommended actions periodically. In the case of the exemplary embodiment, the support system 10B changes at least part of the actions recommended for the user per one week. For example, two of the three recommended actions are replaced by new recommended actions. The new recommended actions are selected from among previously unrecommended actions as much as possible.

### [Step 69]

The support system 10B notifies the information terminal 20 of the updated recommended actions and leaning content or the like.

### [Step 70]

The information terminal 20 records the result of practice for the recommended action and the measurement result of blood pressure value or the like in the auxiliary storage device 104.

### [Step 71]

The information terminal 20 transmits the recorded result of practice or the like to the support system 10B.

Hereinafter, the processing operation corresponding to Steps 68 to 71 is executed every week.

Note that, in the case of the exemplary embodiment, the processes from Step 62 are carried out repeatedly every month. In other words, the featured categories are reviewed and updated on a monthly basis, and the recommended actions to improve the updated featured categories are presented and put into practice repeatedly.

### [Step 7]

Return to the description of FIG. 4.

When a positive result was obtained in the determination of step 5 (i.e., in case of the operation of the “Transferring data” button 203 (refer to FIG. 5)), the support system 10B instructs the information terminal 20 to readout the user data.

### [Step 8]

The information terminal 20 uploads user data stored in the information terminal 20 related to the support service A that is used in the past to the support system 10B. The user data here is an example of the first information.

### [Step 9]

While linking the received user data with the user M and storing, the support system 10B executes a process of transferring that is to register the stored user data in the database of the support service B.

The process of transferring includes the following types.
(a) When the attribute name of the transferring source and the attribute name of the transferring destination are the same, and the expression method of attribute value is also the same.
(b) When the attribute name of the transferring source and the attribute name of the transferring destination are the same, but the expression method of attribute value is different.
(c) When the expression of the attribute name of the transferring source and the attribute name of the transferring destination are different, but there is a record of the corresponding relationship in the user data transferring table 104C (refer to FIG. 2).
(d) When the expression of the attribute name of the transferring source and the attribute name of the transferring destination are different, and there are no records of the corresponding relationship in the user data transferring table 104C.

Firstly, in case of (a), the user data of the transferring source (the first information) is registered as the user data of the transferring destination (the second information) as it is. Secondly, in case of (b), the user data of the transferring source (the first information) is registered as the user data of the transferring destination (the second information) after changing the expression of the user data of the transferring source (the first information).

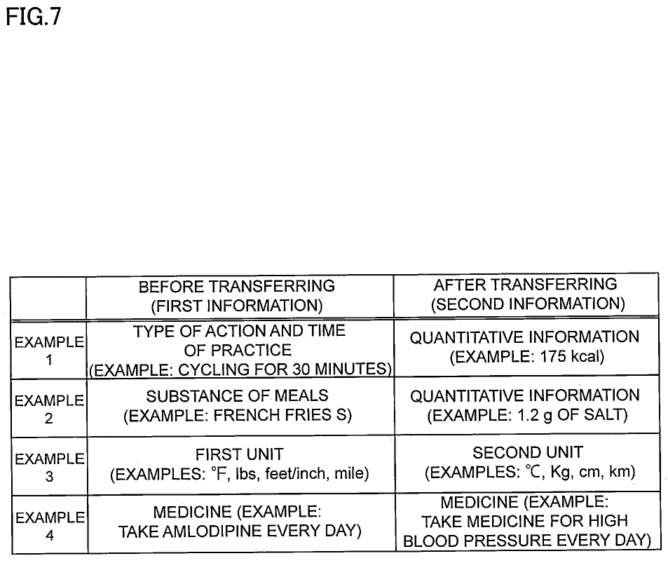

FIG. 7 shows an example of a conversion of expression executed in the process of transferring.

Example 1 represents, an example of converting, for example, a record of the type of action and the time of practice to quantitative information. In conversion example of this type, for example, there is an example that converts a record of cycling for 30 minutes to a expression of consumed calories (for example, exercise of 175 kcal).

Example 2 represents, an example of converting, for example, a record of the substance of the meal expressed in the name of food, to quantitative information. In conversion example of this type, for example, there is an example that converts French fries S to 1.2 g of salt.

Example 3 represents, for example, an example of registering by converting attribute value, according to difference of units before and after transferring. In conversion example of this type, for example, there is an example of converting a temperature, which is expressed in Fahrenheit (first unit), to which is expressed in Celsius (second unit). In addition, for example, there is an example of converting a body weight, which is expressed in pound (the first unit) to kilogram (the second unit). Further, for example, there is an example of converting a body height, which is expressed in feet or inches (the first unit), to which is expressed in centimeters (the second unit). Moreover, for example, there is an example of converting a distance, which is expressed in miles (the first unit), to which is expressed in kilometers (the second unit).

Example 4 represents, an example of registering an attribute value by converting according to the difference of granularity in the expression before and after transferring. In conversion example of this type, for example, there is an example of converting a record of “take Amlodipine every day”, which is a expression in terms of the generic name, to “take medicine for high blood pressure every day”, is a expression in terms of the disease.

In case of (c), the attribute value is transferred to the attribute name of the transferring destination which has corresponding relationship with the attribute name of the transferring source. For example, the attribute value of “blood pressure” (e.g. 136/85 mmHg) is recorded in the attribute value of “blood pressure”.

In case of (d), the user data of the transferring source is excluded from the target of the transferring in this process of transferring.

Note that, when the registration information that is necessary for providing the support service B lacks, the support system 10B requests the information terminal 20 to register the lacked information.

### [Step 10]

Return to the description of FIG. 4.

The support system 10B provides the continuous services using the transferred user data.

### Example 1

For example, when the diagnosis result of lifestyle is transferred, the support system 10B sets, based on the transferred diagnosis result of lifestyle, at least one of the action to be practiced by the target user and the content which encourages to practice the action.

By this setting, the support system 10B is able to provide the action to be practiced by the user M and the content which encourages the user M to practice the action, with high accuracy, immediately after changing job.

Note that, when the diagnosis result of lifestyle is transferred, but not used in the service for the user M, there is need for the support system 10B to restart questions or the like about learning content and lifestyle from scratch.

From considering these questions or the like in terms of the user M, the questions mean forcing to answer duplicately for questions of the same kind which are experienced in previous workplace. Answering to duplicated questions may be workload for the user M, and also may be a cause to heighten dissatisfaction with the support service B.

Therefore, by using the diagnosis result related to lifestyle which is transferred as user data, it is possible to provide the action and content which contribute to improve the user M's lifestyle while reducing workload for the user M.

### Example 2

For example, when the information that motivates the continuous practice of the action is transferred, the support system 10B sets, based on the transferred information, at least one of the difficulty level of an action to be practiced by the target user and the difficulty level of a content which encourages to practice the action.

By this setting, the support system 10B is able to provide the difficulty level of action to be practiced by the user M and the difficulty level of the content which encourages the user M to practice the action, with high accuracy, immediately after changing job.

In the information that motivates the continuous practice of the action, there are a number of completions of the action to be practiced and a point that is given for the number of completions.

In the case of the exemplary embodiment, the difficulty level of actions means, for example, duration for practicing actions, a goal of actions, the size of load of the action, and the difficulty of continuous practice.

For example, when the action to be practiced is “cycling”, the difficulty level of cycling for 30 minutes is higher than the difficulty level of cycling for 10 minutes, and the difficulty level of cycling for 10 km is higher than the difficulty level of cycling for 5 km. In addition, the difficulty level of jogging is higher than the difficulty level of walk, and the difficulty level of practicing every day is higher than the difficulty level of practicing for three days in a week.

For example, when the content which encourages to practice the action is expressed in accumulated point, the accumulated point whose difficulty level is low, for instance, is set to zero point, and the accumulated point whose difficulty level is high, is set to 300 points.

In addition, for example, when the content which encourages to practice the action is an object that imitates plant, the plant whose difficulty level is low is expressed by a seed or a two-leaf plant, while the plant whose difficulty level is high is set to a state in which the plant blooms flowers or bears fruit.

Note that, when the information that motivates the continuous practice of the action is transferred, but not used in the service for the user M, the information is provided to the user M, in a state that the difficulty level of the action to be practiced and the difficulty level of the content which encourages to practice the action are reset to initial value.

However, for example, when the point that has reached 300 points is reset to zero points, or when the plant that is expressed in a state that a flower bloom returns to a state of a seed, there is a risk that willingness to practice the continuous action by the user M is damaged.

Therefore, by using the information that motivates the continuous practice of the action, which is transferred as the user data, it is possible to support the continuous practice of the action which contributes to improve the user M's lifestyle.

Note that, a case is considered that standard is different between the difficulty level of “action to be practiced” provided in the service B and the difficulty level of “action to be practiced” provided in the service A. For example, there is a case that the difficulty level is given according to a scale of one to ten in the service A, while the difficulty level is given according to a scale of one to five or one to twenty in the service B. In this case, the difficulty level of service A is converted to the difficulty level of service B.

In addition, a case is considered that “content that encourages the practice of the actions” provided in the service B is different from “content that encourages the practice of the actions” provided in the service A. For example, there is a case that the content is an object or the like that imitates fish in the service A, while the content is an object or the like that imitates plant in the service B. In this case, the expression of the difficulty level in the service A is converted to the expression of the difficulty level in the service B. For example, the difficulty level expressed by the size of the fish is converted to the difficulty level expressed by the size of the plant.

### Example 3

For example, when a content learned before transferring and a record of proficiency with the content are transferred, the support system 10B sets, based on the transferred information, at least one of the difficulty level of the action to be practiced by the target user and the difficulty level of the content which encourages to practice the action.

By this setting, the support system 10B is able to provide the difficulty level of the action to be practiced by the target user and the difficulty level of the content which encourages to practice the action, with high accuracy, immediately after changing job.

Note that, the learned content refers to, for example, a content that describes disease relates to lifestyle, a content that describes an influence which the improvement of lifestyle gives to health, a content that describes the featured categories which have a high possibility of contributing to improve lifestyle and a content that describes effect or the like which the action to be practiced brings.

In addition, the proficiency with the content is given by, for example, the number of times the content is viewed and the percentage of correct answers to the confirmation questions. The more the number of times the content is viewed and the higher the percentage of correct answers, the higher proficiency is evaluated.

For the user M whose proficiency is high, for example, the difficulty level of the action to be practiced and the difficulty level of the content which encourages the practice of the action are set high.

On the contrary, for the user M whose proficiency is low, for example, the difficulty level of the action to be practiced and the difficulty level of the content which encourages the practice of the action are set low.

By the way, the difficulty level of the content is evaluated by, for example, high volume of information. For example, for the user M whose proficiency is low, a content whose substance is simple is provided. On the other hand, for the user M whose proficiency is high, a content which has a lot of information is provided.

Therefore, by using the proficiency with the content which has already been learned and setting the action recommended for the user M and the difficulty level of the content which encourages the practice of the action, it is possible to support the practice of the action which contributes to improve the user M's lifestyle while reducing workload for the user M.

### Brief Conclusion

According to the information processing system 1B (refer to FIG. 1) by the exemplary embodiment, it is possible to support effective use of information collected by different support app A.

In other words, by transferring the user data before changing job as much as possible, it is possible to reduce workload of registration for new support service B. For example, the user M who is a worker changing job becomes free from inputting duplicate items and answering duplicate questions. As the result, the use M can start use of the new support service B smoothly.

In addition, in the exemplary embodiment, by transferring the information such as the diagnosis results of lifestyle in the support service A before changing job, it is possible to improve quality of service which is provided to the user M from immediately changing job. Specifically, the service provided to the user M by changing job is not initialized. As a result, it becomes to be possible for the user M to improve lifestyle continuously, even when the user moves to a different company or the like, where the adopted support service is different. Consequently, also at a job change destination company or the like, it becomes to be easy to maintain new employee's health, and to be executed improvement of health.

### Exemplary Embodiment 2

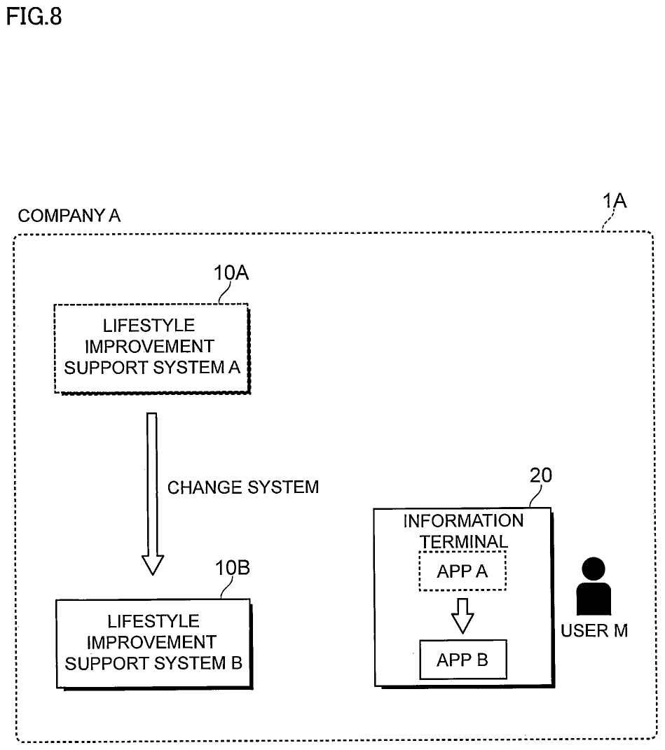

FIG. 8 illustrates a cause of transferring user data assumed in Exemplary embodiment 2. In FIG. 8, components corresponding to those of FIG. 3 are provided with same reference signs.

In FIG. 8, it is assumed that the system that is adopted in the company A which the user M belongs to changes from the support system 10A to the support system 10B. Also in this case, the user M's user data needs to be transferred to the support system 10B that is a new system.

Note that the hardware configurations of the support system 10A and 10B in the exemplary embodiment, and substance of the process of transferring are same as those in Exemplary embodiment 1.

In case of the information processing system 1A in the exemplary embodiment (refer to FIG. 1), as well as the exemplary embodiment 1, it is possible to support effective use of information collected by different support app.

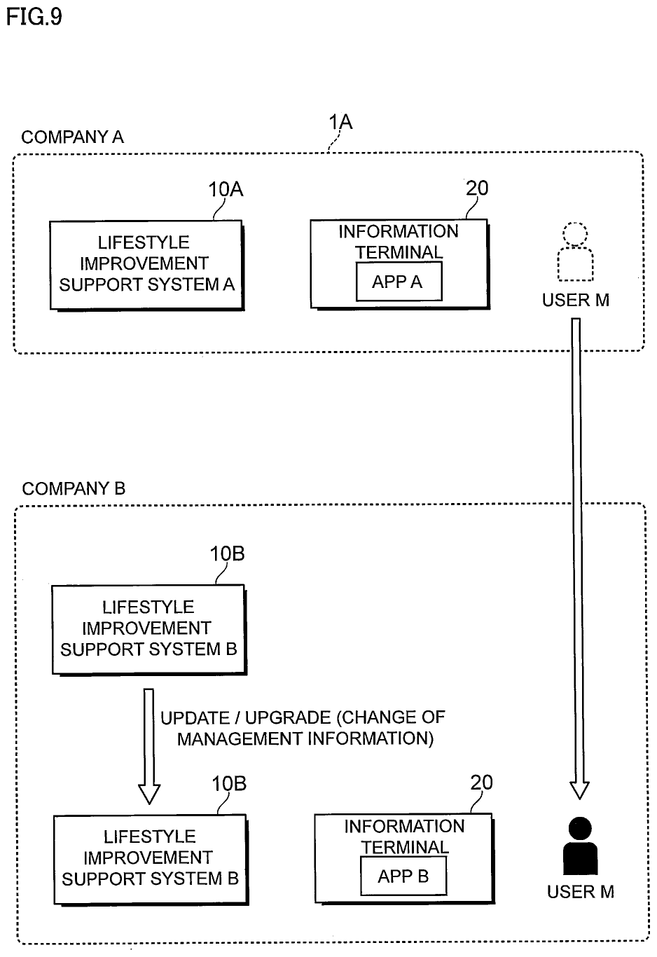

FIG. 9 illustrates a cause of transferring user data assumed in Exemplary embodiment 3. In FIG. 9, components corresponding to those of FIG. 3 and FIG. 8 are provided with same reference signs.

In the example shown in FIG. 9, it is assumed that an update or an upgrade is executed in the support system 10B adopted in the company B after the user M changes job.

More specifically, in the process of transferring when changing the job, it is assumed that the user data which is in holding state without being transferred over to the support system 10B is added to management information in the support system 10B by an update or the like.

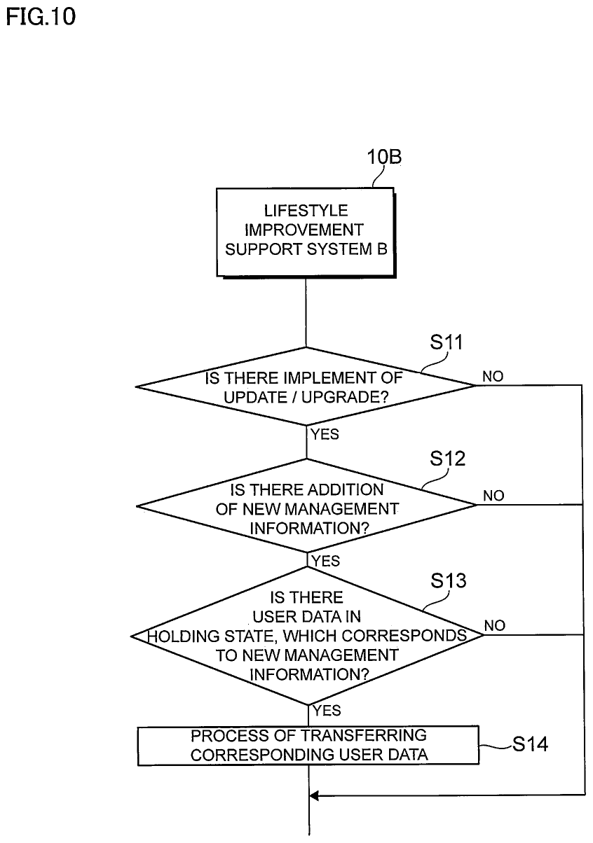

FIG. 10 is a flowchart illustrating a process of transferring user data, which is in holding state.

### [Step 11]

Firstly, the support system 10B determines whether there is an execution of an update or an upgrade.

When a negative result was obtained in the step 11 (i.e., there is not an execution of an update or an upgrade), the support system 10B ends the process shown in FIG. 10, without executing the following processes.

### [Step 12]

When a positive result was obtained in the step 11 (i.e., there is an execution of an update or an upgrade), the support system 10B determines whether it is addition of new management information.

Note that, when a negative result is obtained in the step 12 (i.e., it is not an addition of new management information), the support system 10B ends the process shown in FIG. 10, without executing the following processes.

### [Step 13]

When a positive result was obtained in the step 12 (i.e., there is an addition of new management information), the support system 10B determines whether there is user data in holding state, which corresponds to the new management information.

Note that, when a negative result was obtained in the step 13 (i.e., there is not user data in holding state, which corresponds to the new management information), the support system 10B ends the process shown in FIG. 10, without executing the following processes.

### [Step 14]

When a positive result was obtained in the step 13 (i.e., there is user data in holding state, which corresponds to the new management information), the support system 10B executes the process of transferring the corresponding user data.

Herewith, it becomes to be possible to add user data which is in holding state, since management information which corresponds to the support service A, which is used before changing job, does not have in the support service B, to the support service B's user data at the timing for an update or the like.

Therefore, it becomes to be possible to provide the support service including a history of the user M's previous practices, immediately after an update or the like.

For example, it is assumed that “information that motivates the continuous practice of the action” is managed in the support system 10A before changing job, but is not managed in the support system 10B immediately after changing job.

In this case, “information that motivates the continuous practice of the action” is not in a state ready to be transferred to the support system 10B, but managed as holding state.

However, in the case of the exemplary embodiment, transferring “information that motivates the continuous practice of the action” to the support system 10B is executed at the timing of an update or the like.

As the result, the support system 10B after updated is able to immediately start providing the support service based on “information that motivates the continuous practice of the action” that is transferred from the support system 10A, without waiting an accumulation of the user data which is collected after an update or the like. Specifically, it becomes to be possible to set the difficulty level of the action to be practiced by the user M and the difficulty level of the content which encourages user M to practice the action to the difficulty level which corresponds to the user M from the beginning.

Note that, the example of the management information added newly is not limited to the above-described “information that motivates the continuous practice of the action”, it may be for example, “a diagnosis result of lifestyle”, “an action to be practiced by the target user”, “a content which encourages to practice the action to be practiced by the target user”, “a content learned before transferring”, “a record of proficiency with the content learned before transferring”, “the difficulty level of the action to be practiced by the target user” or “the difficulty level of the content which encourages to practice the action to be practiced by the target user”.

### Other Exemplary Embodiments

(1) So far, the exemplary embodiments of the present disclosure have been described, but the technical scope of the present disclosure is not limited to the scope of the above-described exemplary embodiments. Various modifications or improvements added to the above-described exemplary embodiments may be apparently included in the technical scope of the present disclosure from the description of the claims.

(2) The processor in the above-described exemplary embodiments refers to a processor in a broad sense including, in addition to a general-purpose processor (for example, a Central Processing Unit (CPU)), a dedicated processor (for example, a Graphical Processing Unit (GPU), an Application specific Integrated Circuit (ASIC), a Field Programmable Gate Array (FPGA), a program logic device, etc.).

Moreover, the operation of the processor in each of the above-described exemplary embodiments is not limited to a single processor, but may be performed in cooperation by multiple processors. In addition, the order of execution of each operation in the processor is not limited to the order described in each of the above-described exemplary embodiments, but may be changed individually.

(3) In the above-described exemplary embodiments, it is assumed that the operator of the support system 10A and 10B (refer to FIG. 1) is a corporation or an enterprise entrusted by a corporation, but it may be an enterprise that includes various organizations and institutions. In addition, the operator may also be an enterprise that operates a public medical insurance system or private insurance system, or a medical profession that provides medical services.

(4) The support services in the above-described exemplary embodiments aim to support the prevention, diagnosis, and therapy of high blood pressure, but can also be applied to other diseases.

The support services can be applied to customize messages in services that support the improvement of lifestyle related to, for example, nicotine addiction, insomnia disorder, depression, diabetes, alcoholism, and obesity.

By the way, in the case of nicotine addicted users, for example, carbon monoxide (CO) in exhaled breath, nicotine concentration in saliva, date and time of smoking, the number of cigarettes smoked or the number of times of smoking, user cognitive information (way of thinking, values), exercise (the number of steps, length of exercise time, travel distance), other diseases (diabetes), medications, blood pressure, heart rate, pulse, current physical condition (headache, irritation, feeling good, nausea, etc.), and all or part of the answers for assumed causes are stored as the user data. These information items are examples of information related to smoking.

In addition, in the case of insomnia users, all or part of, for example, bedtime, wake time, sleep hours, sleep efficiency, the number and substance of meals, exercise records, and medications records are stored as the user data. The bedtime, wake time, sleep hours, and sleep efficiency are examples of information related to sleep.

In addition, in the case of depressed users, all or part of, for example, mood records, action records, medications records, sleep records, and appetite records is stored as the user data.

In addition, in the case of users with diabetes, all or part of, for example, the number and substance of meals, exercise records, medications records, weight, blood pressure value, and blood sugar level is stored as the user data. The blood sugar level is an example of information about the blood sugar level.

In addition, in the case of alcoholic users, all or part of, for example, medications records, presence or absence of drinking, and substance of drinking is stored as the user data.

In addition, in the case of users with obesity, all or part of, for example, a target weight, therapy start date, the number of days of therapy, optimum intake calorie, physical condition history (mood), weight history, calorie intake history, calorie intake date and time, medications history (dose, medications date and time), therapy history (therapy, details of advice, presence or absence of self-care by the user), medical care history, blood data (aspartate aminotransferase (AST), alanine aminotransferase (ALT)), and CT values is stored as the user data.

### CONCLUSION

The disclosure examples described in the above-described exemplary embodiments are shown below.

(((1)))

An information processing system including: at least one processor, wherein the at least one processor acquires first information related to lifestyle collected through a first lifestyle improvement support application, and transfers over the first information to second information based on corresponding relationship between the acquired first information and the second information used in a second lifestyle improvement support application.

According to the information processing system, it is possible to support effective use of information collected by a different lifestyle improvement support app.

(((2)))

The information processing system described in (((1))), wherein when a diagnosis result of lifestyle is included in information which is transferred from the first information to the second information, the processor sets, based on the transferred diagnosis result, at least one of an action to be practiced by the target user and a content which encourages to practice the action.

According to the information processing system, it is possible to support improvement of the target user's lifestyle by using the diagnosis result which is before transferring.

(((3)))

The information processing system described in (((1))) or (((2))), wherein when information that motivates a continuous practice of an action is included in information which is transferred from the first information to the second information, the processor sets, based on the transferred information that motivates the continuous practice of the action, at least one of a difficulty level of an action to be practiced by a target user and a difficulty level of a content which encourages to practice the action.

According to the information processing system, it is possible to support improvement of the target user's lifestyle by using information which is before transferring.

(((4)))

The information processing system described in any one of (((1))) to (((3))), wherein when a content learned before transferring and a record of proficiency with the content are included in information which is transferred from the first information to the second information, the processor sets, based on the transferred record, at least one of a difficulty level of an action to be practiced by a target user and a difficulty level of a content which encourages to practice the action.

According to the information processing system, it is possible to support improvement of the target user's lifestyle by using information which is before transferring.

(((5)))

The information processing system described in any one of (((1))) to (((4))), wherein when a record of an action practiced before transferring is included in the first information, the processor converts the record to corresponding quantitative information, transfers the corresponding quantitative information to the second information.

According to the information processing system, it is possible to support improvement of the target user's lifestyle by using information which is before transferring even when the expression form of the first information and the second information is different.

(((6)))

The information processing system described in any one of (((1))) to (((5))), wherein when information related to health in the first information is expressed in a first unit and information related to health in the second information is expressed in a second unit which is different from the first unit, the processor converts an expression of the information related to the health from the first unit to the second unit.

According to the information processing system, it is possible to support improvement of the target user's lifestyle by using information which is before transferring even when the unit used in the expression of information is different between the first information and the second information.

(((7)))

The information processing system described in any one of (((1))) to (((6))), wherein when substance of a target user's diet in the first information is expressed in a food name, the processor converts an expression in the food name to an expression in calorie or nutrient, and transfers the converted expression to the second information.

According to the information processing system, it is possible to support improvement of the target user's lifestyle by using information which is before transferring even when the method of the expression of information is different between the first information and the second information.

(((8)))

The information processing system described in any one of (((1))) to (((7))), wherein when a medication record by a target user is included in the first information, the processor converts an expression of medication record according to granularity expressed in the second information and transfers the converted expression to the second information.

According to the information processing system, it is possible to support improvement of the target user's lifestyle by using information which is before transferring even when granularity of medications records is different between the first information and the second information.

(((9)))

The information processing system described in any one of (((1))) to (((8))), wherein in an update or an upgrade of the second lifestyle improvement support application which is executed after transferring the first information to the second information, when the first information in a holding state becomes a state ready to be transferred, the processor executes transferring the first information in a state ready to be transferred to the second information.

According to the information processing system, it is possible to enable the first information to be transferred, which is in a holding state from transferring due to the difference of information that the lifestyle improvement support application handles at the timing of an update or an upgrade.

(((10)))

The information processing system described in any one of (((1))) to (((9))), wherein when an operation of transferring button is accepted on an operation screen of the second lifestyle improvement support application, the processor executes a process of transferring the first information that is a target for transferring.

According to the information processing system, it is possible to start transferring by an easy operation.

(((11)))

An information processing method that includes acquiring first information related to lifestyle collected through a first lifestyle improvement support application, and transferring the first information to second information, based on corresponding relationship between the acquired first information and the second information used in a second lifestyle improvement support application.

According to the information processing method, it is possible to support effective use of information collected by different lifestyle improvement support application.

Note that, in this information processing method, it is possible to combine technologies corresponding to (((2))) to (((10))).

(((12)))

A non-transitory computer readable medium storing a program that causes a computer to execute an information processing method including; acquiring first information related to lifestyle collected through a first lifestyle improvement support application, and transferring the first information to second information, based on corresponding relationship between the acquired first information and the second information used in a second lifestyle improvement support application.

According to the program, it is possible to support effective use of information collected by different lifestyle improvement support application.

Note that, in this program, it is possible to combine technologies corresponding to (((2))) to (((10))). It is intended that the scope of the invention be defined by the following claims and their equivalents.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The exemplary embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An information processing system comprising:
at least one processor, wherein
the at least one processor acquires first information related to lifestyle collected through a first lifestyle improvement support application, and transfers the first information to second information based on corresponding relationship between the acquired first information and the second information used in a second lifestyle improvement support application.

2. The information processing system according to claim 1, wherein when a diagnosis result of lifestyle is included in information which is transferred from the first information to the second information, the processor sets, based on the transferred diagnosis result, at least one of an action to be practiced by a target user and a content which encourages to practice the action.

3. The information processing system according to claim 1, wherein when information that motivates a continuous practice of an action is included in information which is transferred from the first information to the second information, the processor sets, based on the transferred information that motivates the continuous practice of the action, at least one of a difficulty level of an action to be practiced by a target user and a difficulty level of a content which encourages to practice the action.

4. The information processing system according to claim 1, wherein when a content learned before transferring and a record of proficiency with the content are included in information which is transferred from the first information to the second information, the processor sets, based on the transferred record, at least one of a difficulty level of an action to be practiced by a target user and a difficulty level of a content which encourages to practice the action.

5. The information processing system according to claim 1, wherein when a record of an action practiced before transferring is included in the first information, the processor converts the record to corresponding quantitative information, and transfers the corresponding quantitative information to the second information.

6. The information processing system according to claim 1, wherein when information related to health in the first information is expressed in a first unit and information related to health in the second information is expressed in a second unit which is different from the first unit, the processor converts an expression of the information related to the health from the first unit to the second unit.

7. The information processing system according to claim 1, wherein when substance of a target user's meal in the first information is expressed in a food name, the processor converts an expression in the food name to an expression in calorie or nutrient, and transfers the converted expression to the second information.

8. The information processing system according to claim 1, wherein when a medication record by a target user is included in the first information, the processor converts an expression of the medication record according to granularity expressed in the second information and transfers the converted expression to the second information.

9. The information processing system according to claim 1, wherein in an update or an upgrade of the second lifestyle improvement support application which is executed after transferring the first information to the second information, when the first information in a holding state becomes a state ready to be transferred, the processor executes transferring the first information in a state ready to be transferred to the second information.

10. The information processing system according to claim 1, wherein when an operation of a transferring button is accepted on an operation screen of the second lifestyle improvement support application, the processor executes a process of transferring the first information that is a target for transferring.

11. An information processing method comprising:
acquiring first information related to lifestyle collected through a first lifestyle improvement support application, and
transferring the first information to second information, based on corresponding relationship between the acquired first information and the second information used in a second lifestyle improvement support application.

12. A non-transitory computer readable medium storing a program that causes a computer to execute an information processing method comprising;
acquiring first information related to lifestyle collected through a first lifestyle improvement support application, and
transferring the first information to second information, based on corresponding relationship between the acquired first information and the second information used in a second lifestyle improvement support application.
